(19) United States
(12) Patent Application Publication
(10) Pub. No.: US 2010/0291015 A1
(43) Pub. Date: Nov. 18, 2010

(54) METHOD FOR MAKING UP THE LIPS

(75) Inventors: Claudia Barba, Paris (FR); Aline Abergel, Boulogne (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 12/741,915

(22) PCT Filed: Dec. 19, 2008
(86) PCT No.: PCT/IB2008/055463

§ 371 Date: Jul. 16, 2010

Related U.S. Application Data
(60) Provisional application No. 61/006,521, filed on Jan. 17, 2008.

(30) Foreign Application Priority Data
Dec. 20, 2007 (FR) 0760144

(51) Int. Cl. A61K 8/92; A61Q 1/04

(52) U.S. Cl. 424/64

(57) ABSTRACT

The invention relates to a method for making up the lips comprising the steps of selecting at least one composition, referred to as ‘first composition’, from a range of first compositions formulated in the liquid state, of selecting at least one composition, referred to as ‘second composition’, from a range of second compositions distinct from the range of first compositions, of mixing the first and second compositions at ambient temperature and of applying said mixture to the lips.

The present invention aims at providing a makeup method for the lips capable of providing a personalized makeup.

The personalization of a makeup is a theme which is increasingly seductive to consumers. They are looking for a lipstick which is, on the one hand, specific to them and which, on the other hand, can be coordinated with their appearance or also according to their moods.

Furthermore, it is clear that this personalization of the makeup must not be acquired at the expense of the performance of the makeup proper, namely, for example, satisfactory wearing, comfort, gloss and properties of non transfer and/or of coverage.

In order to respond to this concern for personalization displayed by consumers, databases, in paper form or, generally, in electronic form, have been developed by companies which distribute cosmetic products. These databases give information regarding the ranges of existing products for a given makeup and guide the user towards a specific product from the viewpoint of criteria of choice which she has furthermore provided to this database. For example, by consulting a database of this type, the consumer can choose from a whole range of compounds from the viewpoint of particular features which are specific to her, such as flesh tint of the skin, color of the eyes, color of the hair or morphology of the face.

Nevertheless, this alternative does not always allow the consumer to have available a makeup product which is personal to her and which has a performance and/or makeup effect which can be adjusted at will.

Another alternative, illustrated by U.S. Pat. No. 5,971,351, aims at providing the user with a makeup kit for the lips which combines a palette of makeup bases and a whole range of color pigments. However, the specimen of lipstick is not in this case made immediately available. This is because it is necessary for the user to melt beforehand the makeup base which she has selected and to disperse therein the pigments which she has selected in parallel. The expected lipstick stick is only obtained after the preceding mixture has cooled in a lipstick mold provided for this purpose in the kit.

At the same time, devices for packaging and dispensing at least two, or even more, cosmetic compositions are currently provided which are suitable for the mixing of these compositions in proportions which can be directly adjusted by the consumer. This option allows her to adjust the color and/or optical effect which is personal to her. Such a device is described in particular in the document EP 1 656 853.

However, to the knowledge of the inventors, consumers do not currently have available means which allow them to adjust, on demand and in conjunction with that of the color and/or optical effect, the makeup performance or performances desired.

According to a first aspect, the invention relates to a method for making up the lips comprising the steps consisting in:
selecting at least one composition, referred to as “first composition”, from a range of first compositions, said first compositions being formulated in the liquid state,
selecting at least one composition, referred to as “second composition”, from a range of second compositions, said range of second compositions being distinct from the range of first compositions,
mixing, at ambient temperature, the first and second compositions selected in the preceding stages, and
applying said mixture to the lips.

In particular, the first compositions are differing one from each other by a makeup performance other than a color and/or optical effect.

According to one embodiment, the said differing make up performance is chosen from a wearing, a non-transfer, a comfort, a glossy, a mattness, a coverage, a moisturizing effect, a desquamating effect, or a cleaning effect type performance.

According to a first alternative form, the second compositions can also be formulated in the liquid state.

According to a second alternative form, the second compositions can be formulated in the solid and/or semisolid state.

Within the meaning of the invention, the term “formulation form” is understood to mean the final physical appearance of the composition as it will be used.

Within the meaning of the present invention, a solid or semisolid state characterizes a composition devoid of the ability to flow under its own weight.

It is understood that the method according to the invention is not limited to the use of two ranges of compositions.

According to one embodiment, the user can have available a choice of ranges of compositions extending beyond 2, in particular at least equal to 3, especially at least equal to 4, in particular at least equal to 5, more particularly at least equal to 6, or even more, ranges of compositions.

In all cases, each of the ranges of compositions is devoted to providing a specific makeup performance, the degree of which can be precisely adjusted through the selection of a specific composition constituting the range under consideration.

Thus, through a method of the invention, it becomes possible for the user to adjust, with regard to a single composition, several makeup performances, for example of wearing, of comfort, of gloss or of properties of non-transfer and/or of coverage, to a degree or an intensity more particularly suitable for her.

A method of the invention can employ as many ranges of compositions as makeup performances, the degree of which is to be adjusted.

Furthermore, a range of compositions which is devoted to providing a specific makeup performance, for example a glossy appearance, can comprise at least two, and preferably a number greater than two, specific compositions.

Each of these compositions is advantageously devoted to providing, to a different degree, the makeup performance under consideration.

Thus, such a range can combine compositions which are very different in terms of degree of a given makeup performance, for example of very different degree of gloss.

Thus, according to a specific embodiment, the user can, in a first step, select, from a first range of first compositions, a composition or a composition mixture which makes it possible to adjust the comfort performance to the desired degree.

In a second step, she can, through the selection from a second range of second compositions, more particularly devoted to providing a glossy effect with variable intensities, choose the composition or compositions making it possible to adjust the desired degree of gloss.

The mixture of the two or more preceding compositions can make it possible to have available a specific composition having an expected comfort and an expected glossy effect.

According to a specific embodiment, the user can furthermore adjust the coloring of the composition thus obtained by selecting one or more additional compositions from a third range of third compositions devoted for their part to providing a color effect, for example red, but with different tonalities.

The mixture of the third composition or compositions with the preceding specific composition makes it possible to prepare and to have available a more specific composition corresponding in terms of hue to the expectations of the user.

In such an embodiment, the user can thus formulate, via the method according to the invention, a unique composition adjusted in terms of degree of comfort, of degree of gloss and of degree of hue in every respect in accordance with her expectations.

The first composition(s) employed according to the invention can advantageously be devoid of coloring agent and constitute a “neutral base” which can be white or transparent.

On the other hand, the second compositions comprise at least one material capable of providing a color and/or optical effect.

For example, a second composition can comprise, for example, at least one pearlescent agent, or also at least one coloring agent and one pearlescent agent, or also at least one filler and one coloring agent, or also at least one goniochromatic coloring agent, or a mixture of these.

Furthermore, the first and second compositions can be devoid of UV screening agent. The term “UV screening agent” denotes, within the meaning of the present invention, a material with an absorption spectrum lying mainly in the UV-A and/or UV-B region.

Thus, the invention also has as subject matter, according to another of its aspects, a range of first and/or second compositions.

Advantageously, a range of the invention can be employed in a method of the invention.

The first and second compositions according to the invention comprise a physiologically acceptable medium.

The term “physiologically acceptable medium” denotes a nontoxic medium capable of being applied to human skin, lips or superficial body growths. The physiologically acceptable medium will be suited to the nature of the substrate on which the mixture has to be applied, and to the form under which the compositions are intended to be packaged, for example fluid at ambient temperature and at atmospheric pressure.

First Compositions

A composition referred to as “first composition” according to the invention is a composition of base type capable of providing at least one makeup performance.

A makeup type performance may be, in particular, selected from the group comprising a non-transfer and/or a wearing, in particular a long wearing, a comfort, a glossy, a mattness, a coverage, a moisturizing effect, a desquamating effect or a cleansing effect type performance.

The first compositions can be devoted for providing a single performance or several performances.

In all cases, the group of the first compositions associated with a device according to the invention is composed of compositions which are different from one another. For example, some may be devoted to providing the same performance but to a different degree or with a different scale of intensity.

Wearing

Advantageously, the first compositions according to the invention may be capable of forming a deposited layer having a wearing index of greater than or equal to 65%, or even of greater than or equal to 70%.

The wearing index of the deposited layer obtained with the composition according to the invention can be determined according to the measurement protocol described below.

A support (40 mm×70 mm rectangle) composed of an acrylic coating (hypoallergenic acrylic adhesive on a polyethylene film, sold under the name Blenderm, ref. FH5000-55113, by 3M Health Care) adhesively bonded to a layer of polyethylene foam is prepared which is adhesive on the face opposite that to which the plaster is attached (layer of foam sold under the name RE40X70EP3 by Joint Technique Lyonnais Ind).

The color L*₀a*₀b*₀ of the support, the side of the acrylic coating, is measured using a Minolta CR 300 colorimeter.

The support thus prepared is preheated on a heating plate maintained at a temperature of 40° C. in order for the surface of the support to be maintained at a temperature of 33° C.±1° C. The hot support is removed from the plate and the composition is applied over the entire nonadhesive surface of the support (that is to say, over the surface of the acrylic coating) by spreading it using a brush, in order to obtain a deposited layer of the composition of approximately 15 μm. The combination is placed back on the plate and drying is allowed to take place for 10 minutes.

After drying, the color L*a*b* of the film thus obtained is measured.

The difference in color ΔE1 between the color of the film with respect to the color of the bare support is then determined by the following relationship:

ΔE1=\sqrt{(L*−Lₒ*)²+(a*−aₒ*)²+(b*−bₒ*)²}

The support is subsequently adhesively bonded by its adhesive face (adhesive face of the layer of foam) to an anvil with a diameter of 20 mm provided with a thread. A test specimen of the support/deposited layer combination is subsequently cut out using a hollow punch with a diameter of 18 mm. The anvil is subsequently screwed over a press (Statif Manuel Imada SV-2 from Someco) equipped with a tensile testing device (Imada DPS-20 from Someco).

A strip with a width of 33 mm and a length of 29.7 cm is drawn on a blank photocopier paper with a grammage of 80 g/m², a first line is plotted 2 cm from the edge of the sheet and then a second line is plotted 5 cm from the edge of the sheet, the first and second lines thus delimiting a box on the strip; then a first mark and a second mark situated in the strip are positioned respectively at the points 8 cm and 16 cm from the second line. 20 μl of water are placed on the first mark and 10 μl of refined sunflower oil (sold by Lesieur) are placed on the second mark.

The blank paper is placed over the bed of the press and then the test specimen, placed over the box of the strip of paper, is pressed to a pressure of approximately 300 g/cm² exerted for 30 seconds. Before carrying out a further pressing operation, the test specimen is removed and a measurement corresponding to the postpressing transfer is carried out. The test specimen is subsequently again placed immediately after the second line (thus beside the box), a pressure of approximately 300 g/cm² is again applied and the paper is moved, rectilinearly from the contact produced, with a speed of 1 cm/s over the entire length of the strip, so that the test specimen passes over the deposits of water and of oil.

After removing the test specimen, a portion of the deposited layer has transferred onto the paper. The color L*′a*′b*′ of the deposited layer remaining on the test specimen is then measured.

The difference in color ΔE2 between the color of the deposited layer remaining on the test specimen with respect to the color of the bare support is then determined by the following relationship:

ΔE2=\sqrt{(L*′−Lₒ*)²+(a*′−aₒ*)²+(b*′−bₒ*)²}

The wearing index of the composition, expressed as a percentage, is equal to the ratio

100×ΔE2/ΔE1.

The measurement is carried out on 6 supports in succession and the wearing index corresponds to the mean of the 6 measurements obtained with the 6 supports.

Advantageously, a first composition can have a value for wearing index over time of greater than or equal to 60%, in particular of greater than 65% and especially of greater than 70%.

Non-Transfer

The first compositions according to the invention can also have the ability not to transfer, at least in part, that is to say to leave only lessened marks on certain substrates with which it may be brought into contact, in particular a glass, a cup, a cigarette, a handkerchief, an item of clothing or the skin. The transfer of the cosmetic compositions brings about poor wearing of the film applied, requiring regular renewal of the application of the composition.

The parameter of non-transfer can be measured according to the protocol described below.

The measurements are carried out, for example, on the inner face of the forearm, washed and allowed to dry naturally at ambient temperature for 5 minutes. The cosmetic composition to be tested, for example a lipstick, is applied to three regions of the inner face of the forearm. The skin surface on which the measurements are carried out has to be at least greater than 1 cm². Generally, the measurements are carried out on circular regions with a diameter of approximately 3 cm.

It is necessary for approximately the same amount of cosmetic composition to be applied to each of the three regions. This can be confirmed by measuring the weight of the cosmetic composition, for example the lipstick, after each of the applications or by preparing in advance equivalent amounts of sample to be tested. Generally, for an area of 1 cm², an amount of approximately 2 mg is necessary (if the area has a diameter of 3 cm, then an amount of approximately 28 mg is required).

After application of the cosmetic composition, the color, L₁*a₁*b₁*, of each of the three regions is measured and the mean value obtained corresponds to the initial color of the composition. The color can be measured with a Minolta colorimeter of the CR200 or CR300 or CM500 or CM1000 or CM2000 series. In particular, the Minolta colorimeter of the CR200 series is used.

20 mg/cm² of water are added to each of the three regions to be tested (for areas having a diameter of approximately 3 cm, approximately 280 mg of water have to be applied). Each of the regions to be tested is subsequently subjected to manual massaging for a few seconds, in particular from 2 to 5 seconds and more particularly 2 seconds.

A thickness of a commercial white paper handkerchief, such as Kleenex, the color, L₀*a₀*b₀*, of which was measured, is applied to each made-up region for approximately 5 seconds and at a force of approximately 100 g/f, which force can be applied with a DPZ-5N digital force gauge from the manufacturer Imada Co. Ltd.

The transfer value T is obtained by subtraction of the color of the white tissue measured before application to the region to be tested, L₀*a₀*b₀*, and of the mean color, L₂*a₂*b₂*, corresponding to the mean of the values obtained for each handkerchief after their application to each region of the forearm covered with composition to be tested.

The difference in color ΔE(T) between the color of the handkerchief before and after its application to the region of the forearm carrying the composition is then determined:

ΔE(T)=\sqrt{(L₂*−Lₒ*)²+(a₂*−aₒ*)²+(b₂*−bₒ*)²}

The lower the value ΔE (T) obtained, the more the cosmetic composition is regarded as exhibiting a good level of non-transfer.

Advantageously, the first compositions according to the present invention have a transfer value ΔE (T) of less than or equal to 35 and in particular of less than or equal to 30.

According to one embodiment, a composition referred to as “first composition” can comprise a volatile compound and/or a film-forming agent, optionally with an additional agent which is able to form a film, in an amount sufficient to provide at least a makeup performance of non-transfer and/or wearing type.

Mention may be made, as volatile compound suitable for the invention, of volatile oils, in particular as defined below.

As regards the film-forming agent, it can be chosen from film-forming polymers, as defined below.

According to one embodiment, a composition referred to as “first composition” can have a transfer value of less than or equal to 30 and/or a wearing index of greater than or equal to 60%.

According to another embodiment, the compositions selected from the ranges of compositions of the invention, in particular from a first range of compositions and a second range of compositions, can be such that the mixture applied to the lips can have a transfer value of less than or equal to 30 and/or a wearing index of greater than or equal to 60%.

According to an advantageous embodiment, the first compositions can jointly provide a performance of wearing and/or non-transfer and another performance of comfort type, for example. Thus, the presence of a pasty compound, in particular as defined below, can be advantageous precisely for providing this quality of comfort.

Likewise, a first composition can advantageously jointly provide a glossy nature or, conversely, a matifying nature.

In the first alternative, the first compositions can, for example, comprise one or more glossy oils, in particular as defined below.

In the second alternative, the matifying property can be adjusted by the presence of fillers and/or compounds which absorb sebum, for example as defined below.

The expression of these performances is not incompatible with the joint expression of an optical and/or color effect generally provided by pigments and/or coloring materials.

However, in order to provide the consumer with greater freedom in the personalization of her makeup, it is preferable for these color and/or optical effects to be provided by a composition distinct from that intended to provide the wearing and/or non-transfer performance.

Consequently, the first compositions can advantageously be devoid of pigments and/or coloring materials.

In particular, they can be devoid of titanium oxides, of iron oxides and/or of goniochromatic pigments.

On the other hand, the second compositions forming the device according to the invention comprise at least one pigment and/or one coloring material.

Also, a first composition may advantageously provide a coverage effect.

According to another embodiment, the first compositions may provide a moisturizing type performance.

In such an embodiment, the first compositions may, for example, comprise a moisturizing agent, in particular selected from the group consisting of polyhydric alcohols, hydrosoluble non-ionic alcoxylated polymers, and their mixture.

Preferably, the polyhydric alcohols that may be used are chosen from glycerol, propylene glycol, 1,3-butylen glycol, dipropylen glycol, diglycerol, sodium hyaluronate, and their mixtures.

According to another embodiment, the first compositions may provide a desquamating type performance.

The first compositions may therefore comprise one desquamating agent such as β-hydroxy acid, such as N-octanoyl 5-salycilic acid, urea, glycolic, citric, lactic, tartric, malic or mandelic acid, 4-2(2-hydroxyethyl)piperizine-1-propanesulfonic acid (HEPES), Saphora japonica extract, honey, N-acetylglucosamine, sodium diacetate methylglycine, and their mixtures.

According to another embodiment, the first compositions may provide a sun-screen type performance.

The first compositions may thus comprise a liposoluble UV filter, such as salicylic acid derivatives chosen in the group consisting of homomethyl salicylate, 2-ethylhexyl salicylate, triethanolamine salicylate, 4-isopropylbenzyl salycilate, cinnamic acid derivatives such as 4-methoxy isopentylcinnamate, 4-methoxy 2-ethylhexyl cinnamate, isopropylmethyle cinnamate, 4-methoxyisoamyle cinnamate, 4-methoxy diethanolamine cinnamate, and their mixtures.

Second Composition

A composition referred to as “second composition” of the invention is advantageously capable of providing an optical and/or color effect.

The term “optical effect” denotes an optical effect which can be observed with the naked eye. This optical effect can be a hue effect but also a variation in the color as a function of the angle of observation, the diffraction of the light, the nonhomogeneity in the appearance of the mixture, a volumizing effect, a glossy effect or also mattness, this list not being limiting.

A second composition thus generally comprises at least one material capable of providing a color and/or optical effect.

Such a material can be a coloring agent. The coloring agent(s) under consideration in the compositions according to the invention can be chosen from inorganic pigments, organic pigments or lakes, pearlescent pigments, composite pigments, fat-soluble or water-soluble dyes, and their mixtures.

In the case where the optical effect is coverage or mattness, the second composition can comprise at least one pigment or one filler, in particular a filler which can be chosen from the group consisting of talc, mica, silica, kaolin, sericite, polyamide powders, polyolefin powders, in particular polyethylene powders, polytetrafluoroethylene powders, polymethyl methacrylate powders, polyurethane powders, starch powders and silicone resin beads, and their mixtures.

In the case where the optical effect is the variation in the color as a function of the angle of observation, the second composition can comprise at least one goniochromatic coloring agent, for example chosen from the group consisting of multilayer interference structures and liquid crystal coloring agents.

In the case where the optical effect is the diffraction of light, the second composition can comprise at least one diffractive pigment.

In the case where the optical effect is the nonhomogeneity in the appearance of the mixture, the second composition can comprise colored or reflective particles visible to the naked eye, for example glitter or fibers.

In the case where the optical effect is sparkling or twinkling, the second composition can, for example, comprise reflective particles, pearlescent agents or metal-coated particles, for example according to the formulation form or the destination of the mixture.

According to one embodiment, a composition referred to as “second composition” can comprise at least one pigment and/or one filler in an amount sufficient to provide at least an optical effect of coverage type.

The variation in coverage provided by the second composition can be accompanied, if appropriate, by a variation in the mattness, for example related to the presence of the filler.

By varying the amount of at least one pigment and/or of a filler in the mixture, by virtue of the variation in the relative proportion of the second composition in the mixture, it is possible to obtain a more or less covering mixture, that is to say a mixture exhibiting a more or less high covering power.

Dynamic Color Effect

When at least one optical effect provided by the second composition is the variation of the color as a function of the angle of observation, the second composition can comprise at least one goniochromatic coloring agent.

The term “goniochromatic coloring agent” denotes, within the meaning of the present invention, a coloring agent which makes it possible to obtain, when the composition is spread over a substrate, a color distance in the a*b* plane of the CIE 1976 colorimetric space which corresponds to a variation Dh in the angle of hue h of at least 20° when the angle of observation is varied with respect to the normal by between 0° and 80°, for an angle of incidence of the light of 45°.

The color distance can be measured, for example, using a spectro-gonioreflectometer with the Instrument Systems brand name and with the GON 360 Goniometer reference, after the second composition has been spread in the fluid state with a thickness of 300 μm using an automatic spreader over a contrast chart with the Erichsen brand name and with the Type 24/5 reference, the measurement being carried out on the black background of the chart.

A goniochromatic coloring agent within the meaning of the present invention makes it possible to observe a change in color, also known as color flop, as a function of the angle of observation.

The goniochromatic coloring agent can be chosen, for example, from multilayer interference structures and liquid crystal coloring agents.

In the case of a multilayer structure, the latter can comprise, for example, at least two layers, each layer being produced, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The multilayer structure may or may not exhibit, with respect to a central layer, a symmetry with regard to the chemical nature of the stacked layers. Different effects are obtained according to the thickness and the nature of the various layers.

Examples of symmetrical multilayer interference structures which can be used in compositions produced in accordance with the invention are, for example, the following structures: Fe₂O₃/SiO₂/Fe₂O₃/SiO₂/Fe₂O₃, a pigment having this structure being sold under the name Sicopearl by BASF; MoS₂/SiO₂/mica-oxyde/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the name Xirona by Merck (Darmstadt, Germany).

The liquid crystal coloring agents comprise, for example, silicones or cellulose ethers to which mesomorphic groups are grafted.

Use may be made, as liquid crystal goniochromatic particles, for example, of those sold by Chemx and of those sold under the name Helicone® HC by Wacker.

Use may also be made, as goniochromatic coloring agent, of certain pearlescent agents, effect pigments on a synthetic substrate, in particular a substrate of alumina, silica, borosilicate, iron oxide or aluminum type, or interference holographic glitter resulting from a polyterephthalate film.

The material can additionally comprise dispersed goniochromatic fibers. Such fibers can exhibit a length of less than 80 μm, for example.

The variation in the color as a function of the angle of observation will become more intense as the relative proportion of second composition in the mixture increases.

Diffraction of Light

According to one embodiment, a composition referred to as “second composition” can comprise at least one diffractive pigment in an amount sufficient to provide at least an optical effect of diffraction of light type.

The term “diffractive pigment” denotes, within the meaning of the present invention, a pigment capable of producing a variation in color according to the angle of observation when illuminated by white light, due to the presence of a structure which diffracts the light.

A diffractive pigment can comprise a diffraction grating capable, for example, of diffracting an incident ray of monochromatic light in defined directions.

The diffraction grating can comprise a periodic unit, in particular a line, the distance between two adjacent units being of the same order of magnitude as the wavelength of the incident light.

When the incident light is polychromatic, the diffraction grating will separate the various spectral components of the light and produce a rainbow effect.

Reference may usefully be made, relating to the structure of diffractive pigments, to the paper “Pigments Exhibiting Diffractive Effects” by Alberto Argoitia and Matt Witzman, 2002, Society of Vacuum Coaters, 45th Annual Technical Conference Proceedings 2002.

The diffractive pigment can be produced with units having different profiles, in particular triangular, symmetrical or asymmetrical, square-wave, with a constant or varying width, or sinusoidal.

The spatial frequency of the grating and the depth of the units will be chosen as a function of the degree of separation of the various orders desired. The frequency can vary, for example, between 500 and 3000 lines per mm.

Preferably, the particles of the diffractive pigment each exhibit a flattened form and in particular are in the platelet form.

The same pigment particle can comprise two intersecting, perpendicular or nonperpendicular, diffraction gratings.

The diffractive pigment can exhibit a multilayer structure comprising a layer of a reflective material covered at least on one side with a layer of a dielectric material. The latter may confer better stiffness and durability on the diffractive pigment. The dielectric material can then be chosen, for example, from the following materials: MgF₂, SiO₂, Al₂O₃, AlF₃, CeF₃, LaF₃, NdF₃, SmF₂, BaF₂, CaF₂, LiF and their combinations. The reflective material can be chosen, for example, from metals and their alloys and also from nonmetallic reflective materials. Mention may be made, among the metals which can be used, of Al, Ag, Cu, Au, Pt, Sn, Ti, Pd, Ni, Co, Rd, Nb and Cr, and their materials, combinations or alloys. Such a reflective material can, on its own, constitute the diffractive pigment, which will then be monolayer.

In an alternative form, the diffractive pigment can comprise a multilayer structure comprising a core of a dielectric material covered with a reflective layer at least on one side, indeed even a reflective layer which completely encapsulates the core. A layer of a dielectric material can also cover the reflective layer or layers. The dielectric material used is then preferably inorganic and can be chosen, for example, from metal fluorides, metal oxides, metal sulfides, metal nitrides, metal carbides and their combinations. The dielectric material can be in the crystalline, semicrystalline or amorphous state. In this configuration, the dielectric material can, for example, be chosen from the following materials: MgF₂, SiO, SiO₂, Al₂O₃, TiO₂, WO, AN, BN, B₄C, WC, TiC, TiN, N₄Si₃, ZnS, glass particles and carbons of diamond type, and their combinations.

In an alternative form, the diffractive pigment can be composed of a preformed dielectric or ceramic material, such as a mineral made of natural lamellae, for example peroskovite mica or talc, or synthetic lamellae formed from glass, alumina, SiO₂, carbon, an iron oxide/mica, mica covered with BN, BC, graphite or bismuth oxychloride, and their combinations.

Instead of a layer of a dielectric material, other materials which improve the mechanical properties may be suitable. Such materials can comprise silicone, metal silicides, semiconducting materials formed from elements from Groups III, IV and V, metals having a centered cubic crystal structure, cermet compositions or materials, semiconducting glasses, and their various combinations.

The diffractive pigment used can be chosen in particular from those described in United States patent application US 2003/0031870, published on 13 Feb. 2003, the content of which being incorporated herein by reference.

A diffractive pigment can comprise, for example, the following structure: MgF₂/Al/MgF₂, a diffractive pigment having this structure being sold under the name Spectraflair 1400 Pigment Silver by Flex Products, or Spectraflair 1400 Pigment Silver FG. The proportion by weight of MgF₂ can be between 80 and 95% of the total weight of the pigment.

The amount of diffractive pigment can vary, by weight with respect to the total weight of the second composition, for example from 0.1 to 5%, indeed even from 0.5 to 5%, or also from 0.5% to 2.5%, for example of the order of 1%.

Reflective Particles

The term “reflective particles” denotes, within the meaning of the present invention, particles having a size, a structure, in particular a thickness of the layer or layers of which it is composed and their physical and chemical nature, and a service condition which allow them to reflect incident light. This reflection may, if appropriate, have an intensity sufficient to create, at the surface of the composition or mixture, when the latter is applied to the substrate to be made up, twinkling or highlight points visible to the naked eye, that is to say more luminous points which contrast with their surroundings by appearing to sparkle.

The reflective particles can be selected so as not to detrimentally affect, to a significant extent, the coloring effect generated by the coloring agents which are combined with them and more particularly so as to optimize this effect in terms of color rendition. They can more particularly have a yellow, pink, red, bronze, orangey, brown, gold and/or coppery color or glint.

The reflective particles can be present in the second composition at a content ranging from 0.5% to 60% by weight, with respect to the total weight of the second composition, in particular from 1% to 30% by weight, especially from 2% to 20% by weight, indeed even from 3% to 10% by weight.

These particles can exhibit varied forms and can in particular be in the platelet or globular form, especially the spherical form.

The reflective particles, whatever their form, may or may not exhibit a multilayer structure and, in the case of a multilayer structure, for example at least one layer of uniform thickness, in particular of a reflective material.

When the reflective particles do not exhibit a multilayer structure, they can be composed, for example, of metal oxides, in particular of titanium or iron oxides obtained synthetically.

When the reflective particles exhibit a multilayer structure, they can, for example, comprise a natural or synthetic substrate, in particular a synthetic substrate at least partially coated with at least one layer of a reflective material, in particular of at least one metal or metal material. The substrate can be made of one or more organic and/or inorganic materials.

More particularly, it can be chosen from glasses, ceramics, graphite, metal oxides, aluminas, silicas, silicates, in particular aluminosilicates and borosilicates, synthetic mica and their mixtures, this list not being limiting.

The reflective material can comprise a layer of metal or of a metal material.

Glass particles covered with a metal layer are described in particular in the documents JP-A-09188830, JP-A-10158450, JP-A-10158541, JP-A-07258460 and JP-A-05017710, the content of which being incorporated herein by reference.

Mention may also be made, still by way of example of reflective particles comprising an inorganic substrate coated with a layer of metal, of the particles comprising a borosilicate substrate coated with silver, also known as “white pearlescent agents”.

Particles comprising a glass substrate coated with silver, in the form of platelets, are sold under the name Microglass Metashine REFSX 2025 PS by Toyal. Particles comprising a glass substrate coated with nickel/chromium/molybdenum alloy are sold under the name Crystal Star GF 550 and GF 2525 by this same company.

Reflective particles, whatever their form, can also be chosen from particles comprising a synthetic substrate coated at least partially with at least one layer of at least one metal material, in particular a metal oxide, for example chosen from titanium oxides, in particular TiO₂, iron oxides, in particular Fe₂O₃, tin oxides, chromium oxides, barium sulfate and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅ and MoS₂, and their mixtures or alloys.

Mention may be made, as an example of such particles, for example, of the particles comprising a synthetic mica substrate coated with titanium dioxide, or glass particles coated either with brown iron oxide, with titanium oxide, with tin oxide or with one of their mixtures, such as those sold under the Reflecks® brand name by Engelhard.

The reflective particles may or may not be goniochromatic and/or interference particles.

The second composition according to the invention can comprise at least one pearlescent agent.

Pearlescent Agents

The term “pearlescent agent” should be understood as meaning colored particles of any form, which may or may not be iridescent, produced in particular by certain shellfish in their shells or synthesized, which exhibit a coloring effect by optical interference.

The pearlescent agents can be chosen from pearlescent pigments, such as titanium oxide-coated mica covered with an iron oxide, titanium oxide-coated mica covered with bismuth oxychloride, titanium oxide-coated mica covered with chromium oxide or titanium oxide-coated mica covered with an organic dye, in particular of the abovementioned type, and pearlescent pigments based on bismuth oxychloride. They can also be mica particles, at the surface of which at least two successive layers of metal oxides and/or of organic coloring materials are superimposed.

The pearlescent agents can more particularly have a yellow, pink, red, bronze, orangey, brown, gold and/or coppery color or glint.

Mention may in particular be made, by way of illustration of the pearlescent agents which can be employed in the context of the present invention, of pearlescent agents of gold color sold in particular by Engelhard under the name of Brilliant Gold 212G (Timica), Gold 222C (Cloisonne), Sparkle Gold (Timica), Gold 4504 (Chromalite) and Monarch Gold 233X (Cloisonne); bronze pearlescent agents sold in particular by Merck under the names Bronze Fine (17384) (Colorona) and Bronze (17353) (Colorona) and by Engelhard under the name Super Bronze (Cloisonne); orange pearlescent agents sold in particular by Engelhard under the names Orange 363C (Cloisonne) and Orange MCR 101 (Cosmica) and by Merck under the names Passion Orange (Colorona) and Matte Orange (17449) (Microna); brown-colored pearlescent agents sold in particular by Engelhard under the names Nu-Antique Copper 340XB (Cloisonne) and Brown CL4509 (Chromalite); pearlescent agents with a copper glint sold in particular by Engelhard under the name Copper 340A (Timica); pearlescent agents with a red glint sold in particular by Merck under the name Sienna Fine (17386) (Colorona); pearlescent agents with a yellow glint sold in particular by Engelhard under the name Yellow (4502) (Chromalite); red-colored pearlescent agents with a gold glint sold in particular by Engelhard under the name Sunstone G012 (Gemtone); pink pearlescent agents sold in particular by Engelhard under the name Tan Opale G005 (Gemtone); black pearlescent agents with a gold glint sold in particular by Engelhard under the name Nu-Antique Bronze 240 AB (Timica); blue pearlescent agents sold in particular by Merck under the name Matte Blue (17433) (Microna); white pearlescent agents with a silvery glint sold in particular by Merck under the name Xirona Silver; golden green pinkish orangey pearlescent agents sold in particular by Merck under the name Indian Summer (Xirona); and their mixtures.

Color Effect

Coloring Agents

A second composition can provide a color effect and can comprise at least one coloring agent.

A second composition can comprise a coloring agent in order, for example, to obtain, when mixed with the first composition, a variation in the color and/or in the coverage and/or in another visible optical effect.

The coloring agent can, for example, be chosen from inorganic pigments, organic pigments or lakes, pearlescent pigments, composite pigments, fat-soluble or water-soluble dyes, and their mixtures.

The inorganic pigments can be white or colored and coated or noncoated. Mention may be made of titanium dioxide, optionally treated at the surface, zirconium or cerium oxides, and also iron or chromium oxides, manganese violet, ultramarine blue, chromium hydrate and ferric blue. The pigments can represent from 0 to 40%, preferably from 1 to 35%, better still from 2 to 25%, of the total weight of the composition.

The pearlescent pigments can be chosen from white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride. They can represent from 0 to 20% of the total weight of the composition and better still from 0.1 to 15%, when they are present.

The fat-soluble dyes are, for example, plant extracts, Sudan red, DC Red 17, DC Green 6, β-carotene, soybean oil, Sudan brown, DC Yellow 11, DC Violet 2, DC Orange 5 or quinoline yellow.

The water-soluble dyes are chosen, for example, from plant extracts, in particular beetroot juice and methylene blue.

The dyes can, for example, represent from 0.1 to 20% of the weight of the first composition or of the second composition, indeed even from 0.1 to 6%, when present.

The coloring agent can comprise at least one organic coloring material, for example at least one organic pigment and/or at least one organic lake.

The organic coloring material can be chosen, for example, from particulate materials which are insoluble in the physiologically acceptable medium of the composition.

The organic coloring material can comprise, for example, organic pigments or lakes which can be chosen from the following materials and their mixtures:

cochineal carmine, organic pigments formed of azo, anthraquinone, indigoid, xanthene, pyrene, quinoline, triphenylmethane or fluoran dyes, or organic lakes or insoluble sodium, potassium, calcium, barium, aluminum, zirconium, strontium or titanium salts of acid dyes, such as azo, anthraquinone, indigoid, xanthene, pyrene, quinoline, triphenylmethane or fluoran dyes, it being possible for these dyes to comprise at least one carboxylic or sulfonic acid group.

Mention may in particular be made, among organic pigments, of those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5 and FD&C Yellow No. 6.

The organic coloring material can comprise an organic lake supported by an organic support, such as rosin or aluminum benzoate, for example.

Mention may in particular be made, among organic lakes, of those known under the following names: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake or FD&C Yellow No. 6 Aluminum lake.

The chemicals corresponding to each of the abovementioned organic coloring materials are mentioned in the work “International Cosmetic Ingredient Dictionary and Handbook”, 1997 edition, pages 371 to 386 and 524 to 528, published by “The Cosmetic, Toiletry and Fragrance Association”, the content of which is incorporated in the present patent application by way of reference.

The coloring agent can comprise a composite pigment.

Composite Pigments

The composite pigment can be composed in particular of particles comprising an inorganic core and at least one coating, at least partial, of at least one organic coloring material.

At least one binder can advantageously contribute to the attaching of the organic coloring material to the inorganic core.

The composite pigment particles can exhibit varied forms. These particles can in particular be in the platelet or globular, in particular spherical, form and be hollow or solid. The term “in the platelet form” denotes particles having a ratio of the greatest dimension to the thickness of greater than or equal to 5.

A composite pigment can, for example, exhibit a specific surface of between 1 and 1000 m²/g, in particular between 10 and 600 m²/g approximately and especially between 20 and 400 m²/g approximately. The specific surface is the value measured by the BET method.

The second composition can comprise one or more composite pigments as defined above.

The inorganic core of the composite pigment can be of any form suitable for the attaching of particles of organic coloring material, for example spherical, globular, granular, polyhedral, acicular, spindle-shaped, flattened in the fleck, rice grain or flake form, and a combination of these forms, this list not being limiting.

The ratio of the greatest dimension of the core to its smallest dimension can be between 1 and 50.

The inorganic core can exhibit a size of between approximately 1 nm and approximately 100 nm, indeed even between approximately 5 nm and approximately 75 nm, for example between approximately 10 nm and approximately 50 nm.

The inorganic core can be made of a material chosen from the nonlimiting list consisting of metal salts and metal oxides, in particular titanium, zirconium, cerium, zinc, iron, aluminum and chromium oxides, ferric blue, aluminas, glasses, ceramics, graphite, silicas, silicates, in particular aluminosilicates and borosilicates, synthetic mica and their mixtures.

Titanium oxides, in particular TiO₂, iron oxides, in particular Fe₂O₃, cerium oxides, zinc oxides, aluminum oxides or silicates, in particular aluminosilicates and borosilicates, are very particularly suitable.

The inorganic core can be colored, if appropriate.

The organic coloring material can be as defined above.

The binder of the composite pigment can be of any type, provided that it allows the organic coloring material to adhere to the surface of the inorganic core.

The binder can in particular be chosen from a nonlimiting list consisting of silicone materials, polymeric, oligomeric or similar materials, in particular organosilanes, fluoroalkylated organosilanes and polysiloxanes, for example polymethylhydrosiloxane, and various coupling agents, such as coupling agents based on silanes, on titanates, on aluminates or on zirconates, and their mixtures.

Physiologically Acceptable Medium

The first and second compositions comprise, because of their destination, a physiologically acceptable medium.

The term “physiologically acceptable medium” denotes a nontoxic medium capable of being applied to the human lips. The physiologically acceptable medium is generally suitable for the nature of the substrate on which the composition has to be applied and for the appearance under which the composition is intended to be packaged.

The physiologically acceptable medium can comprise an aqueous phase and/or a fatty phase.

According to a specific embodiment, the aqueous phase or the fatty phase can form the continuous phase of the composition.

Within the meaning of the present invention, the emulsions comprise a lipophilic phase and a hydrophilic phase, the latter not automatically being water.

Thus, the cosmetic compositions in accordance with the invention can be in the form of a water-in-oil, oil-in-water, multiple or anhydrous emulsion.

Likewise, the cosmetic compositions in accordance with the invention can be in the form of anhydrous emulsion.

In particular, a composition can have, for example, a continuous fatty phase which can comprise less than 10% by weight of water, in particular less than 5% by weight of water, especially less than 3% by weight of water and more particularly less than 1% by weight of water, with respect to the total weight of the composition.

The cosmetic compositions according to the invention are advantageously anhydrous, that is to say being able to comprise less than 5% of water, in particular less than 3% of water, in particular less than 2% of water and more particularly less than 1% of water, with respect to the total weight of the composition. They can then be provided in particular in the form of oily gels, of oily liquids, of pastes or of sticks or also in the form of vesicular dispersions comprising ionic and/or nonionic liquids.

Aqueous or Organic Solvents

A first and/or a second composition can comprise at least one aqueous or organic solvent.

When a first and/or a second composition comprises one or more aqueous or organic solvents, these solvents can be present in a content ranging from 0.1 to 99%, with respect to the total weight of the composition concerned.

Generally, the amount of solvent(s), in particular organic solvent(s), will depend on the nature of the substrate on which the composition is intended to be applied.

The aqueous phase can be composed essentially of water or can be formed of a mixture of water and of water-miscible organic solvent (miscibility in water of greater than 50% by weight at 25° C.), such as lower monoalcohols having from 1 to 5 carbon atoms, for example ethanol or isopropanol, glycols having from 2 to 8 carbon atoms, such as propylene glycol, ethylene glycol, 1,3-butylene glycol or dipropylene glycol, C₃-C₄ ketones and C₂-C₄ aldehydes.

Fatty Phase

A first and/or a second composition can comprise a fatty phase and in particular at least one fatty substance which is liquid at ambient temperature (20-25° C.) and at atmospheric pressure and/or a fatty substance which is solid at ambient temperature, such as waxes, pasty fatty substances, gums and their mixtures. The fatty phase can additionally comprise lipophilic organic solvents.

A first and/or a second composition can exhibit, for example, a continuous fatty phase which can comprise less than 5% of water, in particular less than 1% of water, with respect to its total weight and can in particular be in the anhydrous form.

The compositions in accordance with the present invention can comprise a fatty phase comprising in particular oils and/or fatty substances which are solid at ambient temperature (20-25° C.) and atmospheric pressure.

The liquid fatty phase can also comprise, in addition to the oils, other compounds dissolved in the oils, such as gelling and/or structuring agents.

The oil or oils can be present in a proportion of 0.1 to 99% by weight, in particular of at least 1 to 90% by weight, more particularly of 5 to 70% by weight, especially of 10 to 60% by weight, indeed even of 20 to 50% by weight, with respect to the total weight of the composition.

The term “oil” is understood to mean any fatty substance in the liquid form at ambient temperature (20-25° C.) and at atmospheric pressure.

The oils suitable for the preparation of a composition according to the invention can be volatile or nonvolatile and silicone or nonsilicone oils.

Within the meaning of the present invention, the term “nonvolatile oil” is understood to mean an oil having a vapor pressure of less than 0.13 Pa. The volatile or nonvolatile oils can be hydrocarbon oils, in particular of animal or vegetable origin, synthetic oils, silicone oils, fluorinated oils or their mixtures.

Within the meaning of the present invention, the term “volatile oil” is understood to mean an oil (or non-aqueous medium) capable of evaporating on contact with the skin in less than one hour at ambient temperature and at atmospheric pressure. The volatile oil is a volatile cosmetic oil which is liquid at ambient temperature and which has in particular a non zero vapor pressure at ambient temperature and atmospheric pressure, especially a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Within the meaning of the present invention, the term “silicone oil” is understood to mean an oil comprising at least one silicon atom and in particular at least one Si—O group.

The term “hydrocarbon oil” is understood to mean an oil comprising mainly hydrogen and carbon atoms and optionally oxygen, nitrogen, sulfur and/or phosphorus atoms.

The volatile hydrocarbon oils can be chosen from the abovementioned oils, hydrocarbon oils having from 8 to 16 carbon atoms and in particular branched C₈-C₁₆ alkanes (also referred to as isoparaffins), such as isododecane (also referred to as 2,2,4,4,6-pentamethylheptane), isodecane, isohexadecane and, for example, the oils sold under the Isopar® or Permethyl® trade names.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 10 silicon atoms, especially from 2 to 7 silicon atoms, these silicones optionally comprising alkyl or alkoxy groups having from 1 to 10 carbon atoms. Mention may in particular be made, as volatile silicone oil which can be used in the invention, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Use may also be made of volatile fluorinated oils, such as nonafluoromethoxybutane or perfluoromethylcyclopentane, and their mixtures.

It is also possible to use a mixture of the abovementioned oils.

A composition of the invention can also comprise at least one nonvolatile oil.

The nonvolatile oils can be chosen in particular from nonvolatile hydrocarbon oils, if appropriate fluorinated, and/or nonvolatile silicone oils.

Mention may in particular be made, as nonvolatile hydrocarbon oil, of:
hydrocarbon oils of animal origin,
hydrocarbon oils of vegetable origin, such as phytosteryl esters, for example phytosteryl oleate, phytosteryl isostearate and lauroyl/octyldodecyl/phytosteryl glutamate (Ajinomoto, Eldew PS203), triglycerides composed of esters of fatty acids and of glycerol, the fatty acids of which can have varied chain lengths from C₄ to C₂₄, it being possible for these chains to be linear or branched and saturated or unsaturated; these oils are in particular heptanoic or octanoic triglycerides; wheat germ, sunflower, grape seed, sesame, corn, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel,
synthetic ethers having from 10 to 40 carbon atoms;
linear or branched hydrocarbons of mineral or synthetic origin, such as liquid petrolatum, polydecenes, hydrogenated polyisobutene, such as Parleam, squalane and their mixtures, and in particular hydrogenated polyisobutene,
synthetic esters, such as oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂≧10.

The esters can in particular be chosen from esters, in particular fatty acid esters, such as, for example:
cetearyl octanoate, esters of isopropyl alcohol, such as Purcellin oil, isopropyl myristate or isopropyl palmitate, ethyl palmitate, 2-ethylhexyl palmitate, isopropyl stearate or isostearate, isostearyl isostearate, octyl stearate, hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and their mixtures, C₁₂ to C₁₅ alkyl benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, or hydroxylated esters, such as isostearyl lactate or diisostearyl malate;
esters of polyols and esters of pentaerythritol, such as dipentaerythritol tetrahydroxystearate/tetraisostearate,
esters of dimer diols and dimer diacids, such as Lusplan DD-DA5® and Lusplan DD-DA7®, sold by Nippon Fine Chemical and described in application FR 2 851 915 filed on Mar. 6, 2003, the content of which is incorporated in the present patent application by way of reference,
fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol,
higher fatty acids, such as oleic acid, linoleic acid, linolenic acid and their mixtures, and
dialkyl carbonates, it being possible for the 2 alkyl chains to be identical or different, such as dicaprylyl carbonate, sold under the name Cetiol CC® by Cognis.

The nonvolatile silicone oils which can be used in the composition according to the invention can be nonvolatile polydimethylsiloxanes (PDMSs), polydimethyl-siloxanes comprising pendent alkyl or alkoxy groups and/or alkyl or alkoxy groups at the ends of the silicone chain, which groups each have from 2 to 24 carbon atoms, phenylated silicones, such as phenyl trimethicones, phenyl dimethicones, phenyl(trimethylsiloxy)di-phenylsiloxanes, diphenyl dimethicones, diphenyl(methyldiphenyl)trisiloxanes and (2-phenylethyl)trimethylsiloxysilicates, dimethicones or phenyl trimethicones with a viscosity of less than or equal to 100 cSt, and their mixtures.

At least one nonvolatile oil can be present in a composition according to the invention in a content ranging from 20% to 99% by weight, in particular from 30% to 80% by weight and especially from 40% to 80% by weight, with respect to the total weight of the composition.

According to one embodiment, a composition and in particular a second composition can comprise a glossy oil in an amount sufficient to provide at least a makeup performance of gloss type.

Thus, a composition according to the invention can comprise an oil with a high molar mass ranging from 650 to 10 000 g/mol intended to confer gloss properties on the composition. The term “oil” is understood to mean a nonaqueous water-immiscible compound which is liquid at ambient temperature (25° C.) and atmospheric pressure (760 mmHg).

An oil suitable for the formulation of a composition according to the invention can have a molar mass varying from 650 to 10 000 g/mol and in particular from 750 to 7 500 g/mol.

Oils with an excessively low molar mass generally result in compositions which are not very glossy; oils having an excessively high molar mass for their part give compositions regarded as excessively tacky.

An oil with a molar mass ranging from 650 to 10 000 g/mol which can be used in a composition of the invention can be chosen from:
lipophilic polymers, such as:
   polybutylenes, such as Indapol H-100 (with a molar mass or MM=965 g/mol), Indapol H-300 (MM=1340 g/mol) or Indapol H-1500 (MM=2160 g/mol), sold or manufactured by Amoco,
   hydrogenated polyisobutylenes, such as Panalane H-300 E, sold or manufactured by Amoco (M=1340 g/mol), Viseal 20 000, sold or manufactured by Synteal (MM=6000 g/mol), or Rewopal PIB 1000, sold or manufactured by Witco (MM=1000 g/mol),
   polydecenes and hydrogenated polydecenes, such as: Puresyn 10 (MM=723 g/mol) or Puresyn 150 (MM=9200 g/mol), which are sold or manufactured by Mobil Chemicals,
   vinylpyrrolidone copolymers, such as: the vinylpyrrolidone/1-hexa-decene copolymer, Antaron V6216, sold or manufactured by ISP (MM=7300 g/mol),
esters, such as:
   esters of linear fatty acids having from 35 to 70 carbon atoms, such as pentaerythrityl tetrapelargonate (MM=697.05 g/mol),
   hydroxylated esters, such as polyglycerol-2 triisostearate (MM=965.58 g/mol),
   aromatic esters, such as tridecyl trimellitate (MM=757.19 g/mol),
   C₂₄-C₂₈ branched fatty acid or fatty alcohol esters, such as those described in application EP-A-0 955 039 and in particular triisoarachidyl citrate (MM=1033.76 g/mol), pentaerythrityl tetraisononanoate (MM=697.05 g/mol), glyceryl triisostearate (MM=891.51 g/mol), glyceryl tri(2-decyltetradecanoate) (MM=1143.98 g/mol), pentaerythrityl tetraisostearate (MM=1232.04 g/mol) or pentaerythrityl tetra(2-decyltetradecanoate) (MM=1538.66 g/mol),
silicone oils, such as phenylated silicones, for example Belsil PDM 1000 from Wacker (MM=9000 g/mol),
oils of vegetable origin, such as sesame oil (820.6 g/mol),
and their mixtures.

A composition according to the invention can also comprise at least one compound chosen from waxes, pasty fatty substances and their mixtures.

Pasty Compounds

The term “pasty” is understood to mean, within the meaning of the present invention, a fatty compound having a reversible solid/liquid change in state and comprising, at a temperature of 23° C., a liquid fraction and a solid fraction. The term “pasty” is also understood to mean polyvinyl laurate.

A pasty compound within the meaning of the invention can exhibit a hardness at 20° C. ranging from 0.001 to 0.5 MPa, preferably from 0.002 to 0.4 MPa.

Mention may be made, among the pasty compounds capable of being used in a composition of the invention, of lanolins and lanolin derivatives, such as acetylated lanolins, oxypropylenated lanolins or isopropyl lanolate, and their mixtures. Use may also be made of esters of fatty acids or of fatty alcohols, in particular those having from 20 to 65 carbon atoms, such as triisostearyl or cetyl citrate; arachidyl propionate; polyvinyl laurate; cholesterol esters, such as triglycerides of vegetable origin, for example hydrogenated vegetable oils, viscous polyesters and their mixtures. Use may be made, as triglycerides of vegetable origin, of derivatives of hydrogenated castor oil, such as “Thixinr®” from Rheox.

Mention may also be made of polyesters resulting from the esterification of a carboxylic acid and of an aliphatic hydroxycarboxylic acid ester, for example Risocast® DA-L (ester resulting from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3), sold by the Japanese company Kokyu Alcohol Kogyo.

Mention may be made, as pasty compound suitable for the formulation of a composition of the invention, of hydrogenated cocoglycerides.

Mention may also be made of pasty silicone compounds, such as polydimethylsiloxanes (PDMSs) with high molecular weights and in particular those having pendent chains of the alkyl or alkoxy type having from 8 to 24 carbon atoms and a melting point of 20-55° C., such as stearyl dimethicones, in particular those sold by Dow Corning under the trade names of DC2503® and DC25514®, and their mixtures.

The pasty compounds mentioned can be used alone or as a mixture.

Wax

Within the meaning of the invention, a wax is a compound which is solid at ambient temperature (25° C.), which exhibits a reversible solid/liquid change in state, which has a melting point of greater than 30° C. which can range up to 200° C., which has a hardness of greater than 0.5 MPa and which exhibits, in the solid state, an anisotropic crystalline arrangement.

It can be a hydrocarbon, fluorinated and/or silicone wax and be of animal, vegetable, mineral or synthetic origin.

It can be chosen, for example, from beeswax, carnauba wax, candelilla wax, paraffin waxes, hydrogenated castor oil, synthetic waxes, such as polyethylene waxes (preferably with a molecular weight of between 400 and 600) or Fischer-Tropsch waxes, silicone waxes, such as alkyl or alkoxy dimethicones having from 16 to 45 carbon atoms, ceresins or ozokerites, such as, for example, isoparaffins having a melting point of less than 40° C., such as EMW-0003, sold by Nippon Seirou, α-olefin oligomers, such as the Performa V® 825, 103 and 260 polymers sold by New Phase Technologies; ethylene/propylene copolymers, such as Performalene® EP 700, and microcrystalline waxes having a melting point of greater than 85° C., such as HI-MIC® 1070, 1080, 1090 and 3080, sold by Nippon Seirou, and their mixtures.

According to one embodiment, the wax or waxes used in a composition in accordance with the present invention is or are present in a content varying from approximately 1.5 to approximately 20% by weight, in particular from approximately 3 to approximately 15% by weight, in particular from approximately 5 to approximately 10% by weight, with respect to the total weight of the composition.

A composition can also comprise a gum.

The gums which can be used are generally polydimethylsiloxanes (PDMSs) of high molecular weight or cellulose gums or polysaccharides.

Film Forming Polymer

A composition according to the invention can also comprise, if appropriate, a film-forming polymer.

Mention may be made, among the film-forming polymers which can be used in the compositions of the present invention, of synthetic polymers, of radical type or of polycondensate type, polymers of natural origin, and their mixtures.

The film-forming polymers of radical type can in particular be vinyl polymers or copolymers, in particular acrylic polymers, and more particularly (meth)acrylic acid esters, such as alkyl (meth)acrylates, or vinyl esters, such as vinyl acetate, vinyl neodecanoate, vinyl pivalate, vinyl benzoate and vinyl t-butylbenzoate.

Mention may be made, among film-forming polycondensates, of polyurethanes, polyesters, polyesteramides, polyamides, epoxy ester resins or polyureas.

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

Mention may be made, as examples of commercially available polymethylsilsesquioxane resins, of those which are sold:
by Wacker under the reference Resin MK, such as Belsil PMS MK,
by Shin-Etsu under the reference KR-220L.

Mention may be made, as siloxysilicate resins, of trimethylsiloxysilicate (TMS) resins, such as that sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above with polydimethylsiloxanes, such as the pressure-sensitive adhesive copolymers sold by Dow Corning under the reference BIO-PSA and described in the document U.S. Pat. No. 5,162,410, the content of which being incorporated herein by reference, or the silicone copolymers resulting from the reaction of a silicone resin, such as that described above, and of a diorganosiloxane, as are described in the document WO 2004/073626, the content of which being incorporated herein by reference.

Use may also be made of silicone polyamides of the polyorganosiloxane type, such as those described in the documents U.S. Pat. No. 5,874,069, U.S. Pat. No. 5,919,441, U.S. Pat. No. 6,051,216 and U.S. Pat. No. 5,981,680, the content of which being incorporated herein by reference.

In addition to the abovementioned compounds, a first and/or a second composition according to the invention can comprise, according to the type of application envisaged, the constituents conventionally used in the fields under consideration, in an amount appropriate to the formulation form desired.

Thus, a first and/or a second composition can also comprise ingredients commonly used in cosmetics, such as, for example, thickeners, surfactants, fillers, sebum-absorbing compounds, trace elements, moisturizing agents, softeners, sequestering agents, fragrances, basifying or acidifying agents, preservatives, antioxidants, UV screening agents, colorants or their mixtures.

Mention may be made, as examples of fillers, without implied limitation, of inorganic fillers (ZnOSiO₂, and the like), which may or may not be coated, talc, mica, silica, kaolin, sericite, polyamide powders, polyolefin powders, for example polyethylene powders, polytetrafluoroethylene powders, polymethyl methacrylate powders, polyurethane powders, starch powders and silicone resin beads, and their mixtures.

Mention may be made, by way of representation and without implied limitation of the sebum-absorbing and/or sebum-adsorbing compounds according to the invention, of:
polyamide (Nylon®) powders, such as, for example, “Orgasol® 2002 D NAT COS”, sold by Atofina,
acrylic polymer powders, in particular polymethyl methacrylate/ethylene glycol dimethacrylate powders, such as, for example, “Dow Corning 5640 Microsponge® Skin Oil Adsorber”, sold by Dow Corning, or “Micropearl M 305”, sold by Matsumoto Yushi; polyallyl methacrylate/ethylene glycol dimethacrylate powders, such as, for example, “Poly-Pore® E200”, sold by Amcol; or ethylene glycol dimethacrylate/lauryl methacrylate copolymer powders, such as, for example, “Polytrap® 6603”, sold by RP Scherer, and
crosslinked polyalkylstyrene powders, such as “Imbiber Beads 295”, sold by Imbibitive Technologies.

The sebum-absorbing or sebum-adsorbing compound can represent from 0.1 to 10% by weight and preferably from 1 to 5% by weight, with respect to the total weight of a composition.

A first and/or a second composition can also comprise at least one cosmetic or dermatological active principle. Mention may be made, as cosmetic, dermatological, hygienic or pharmaceutical active principles which can be used in the compositions of the invention, of moisturizing agents (of polyol type, such as glycerol), vitamins (C, A, E, F, B or PP), essential fatty acids, essential oils, ceramides, sphingolipids, fat-soluble sunscreens, or sunscreens in the form of nanoparticles, specific active principles for the treatment of the skin (protective agents, antibacterials, antiwrinkle agents, and the like) or self-tanning agents.

These active principles can be used, for example, at concentrations of 0 to 20% and in particular of 0.001 to 15%, with respect to the total weight of the composition.

Formulation Forms

A method of the invention employs at least two ranges of compositions, at least one first range of compositions of which comprises first compositions formulated in the liquid state.

For the compositions according to the invention constituting the additional range or ranges of compositions to be employed, in particular the range of second compositions, it is possible to envisage different formulation forms.

These compositions can thus be provided in any formulation form normally used for a topical application, in particular in the anhydrous form, in the form of an oily or aqueous solution, of an oily or aqueous gel, of an oil-in-water, water-in-oil, wax-in-water or water-in-wax emulsion, of a multiple emulsion or of a dispersion of oil in water by virtue of vesicles situated at the oil/water interface.

According to one embodiment, a first composition and a second composition have a similar formulation form. Thus, for example, a first composition and a second composition can be liquids.

According to another embodiment, the additional range(s) of compositions and in particular the second range of second compositions can comprise compositions formulated in the solid state.

They can be compositions of stick type or of gloss type.

The makeup composition is prepared by combining at least one first composition with at least one second composition using the appropriate device. The two types of compositions are advantageously mixed at ambient temperature.

Advantageously, the preparation of a makeup composition by mixing at least one first composition with a second composition does not necessarily require a heating stage and can thus advantageously be carried out at ambient temperature, which, for obvious reasons, constitutes an advantage in terms of convenience for the consumer.

As specified above, another subject matter of the invention is, according to another of its aspects, a range of first and/or second compositions.

Within the meaning of the invention, a range of compositions brings together at least two different compositions, preferably more than five different compositions, indeed even more. The latter can be employed in a device according to the invention which makes it possible, if appropriate, to mix them in a chosen proportion.

For example, the range of second compositions can comprise second compositions having different colors. Thus, for a given first composition, it will be possible, through the choice of one or more second compositions, brought together in a range of second compositions, to access a great diversity of colors.

Likewise, the range of second compositions can, for example, comprise compositions displaying optical effects of reflective type.

In this case, it will be possible, through the choice of these second compositions, to obtain different optical effects by mixing with a colorless first composition.

Assuming that the range of combined first compositions would bring together colored first compositions, it will be possible for the consumer to access a great diversity of color effects and glossy glint effects by the choice which will appear appropriate to her of the first composition or compositions and second composition or compositions to be combined.

The examples which appear below are presented by way of illustration and without implied limitation of the scope of the invention.

### EXAMPLES

The following makeup formulations can be obtained by mixing at least one first composition, described as white base in the table below, with a second composition described as colored base.

The respective constitutions of the group of the compositions are specified below. The compositions can be obtained according to the methods conventionally used in cosmetics.

Each formulation is obtained by mixing the two types of compositions selected at ambient temperature.

LIQUID FORMULATIONS

| | | |
|---|---|---|
| Gloss Formulation | White Base A1 | Colored Base A2 |
| Long-lasting wearing | White Base B1 | Colored Base B2 |
| Formulation | | |
| Matt Formulation* | White Base C1 | Colored Base C2 |
| Transfer-Free Formulation | White Base D1 | Colored Base D2 |

SOLID FORMULATIONS

| | | |
|---|---|---|
| Comfort Formulation | White Base E1 | Colored Base E2 |
| Long-lasting wearing | White Base F1 | Colored Base F2 |
| Formulation | | |

### Example 1

### Gloss Formulation

White Base A1

| Starting material | % * |
|---|---|
| SQUALANE | 3.86 |
| BHT | 0.06 |
| ISOSTEARYL ALCOHOL | 0.32 |
| TRIOLEYL PHOSPHATE | 0.83 |
| ISOPROPYLPARABEN | 0.13 |
| ISOBUTYLPARABEN | 0.10 |
| OCTYLDODECANOL | 8.28 |
| ETHYLHEXYL METHOXYCINNAMATE | 6.99 |
| GLYCERIN | 6.21 |
| MALIC ACID | 0.10 |
| TRIHEPTANOIN | 3.16 |
| PENTAERYTHRITYL TETRAISOSTEARATE | 6.10 |
| DIISOSTEARYL MALATE | q.s. for 100 |
| SILICA DIMETHYL SILYLATE | 6.21 |
| POLYGLYCERYL-2 TRIISOSTEARATE | 8.86 |
| SILICA | 6.18 |
| BEESWAX CERA ALBA | 5.66 |
| BUTYLPARABEN | 0.10 |
| C₂₃₋₄₃ ACID PENTAERYTHRITOL TETRAESTER | 0.79 |
| | 100 |

Colored Base A2

| Starting material | % |
|---|---|
| SQUALANE | 3.86 |
| MICA | 0.63 |
| BHT | 0.06 |
| IRON OXIDES CI 77491 | 4.00 |
| TITANIUM DIOXIDE CI | 0.20 |
| 77891 | |
| TIN OXIDE | 0.01 |
| ISOSTEARYL ALCOHOL | 0.32 |
| TRIOLEYL PHOSPHATE | 0.83 |
| ISOPROPYLPARABEN | 0.13 |
| ISOBUTYLPARABEN | 0.10 |
| OCTYLDODECANOL | 8.28 |
| ETHYLHEXYL | 6.99 |
| METHOXYCINNAMATE | |
| GLYCERIN | 6.21 |
| MALIC ACID | 0.10 |
| TRIHEPTANOIN | 3.16 |
| PENTAERYTHRITYL | 6.10 |
| TETRAISOSTEARATE | |
| DIISOSTEARYL MALATE | q.s. for 100 |
| SILICA DIMETHYL | 6.21 |
| SILYLATE | |
| POLYGLYCERYL-2 | 8.86 |
| TRIISOSTEARATE | |
| SILICA | 6.18 |
| BEESWAX CERA ALBA | 5.66 |
| BUTYLPARABEN | 0.10 |
| C₂₃-C₄₃ ACID | 0.79 |
| PENTAERYTHRITOL | |
| TETRAESTER | |
| | 100 |

### Example 2

### Long-Lasting Wearing Formulation in the Liquid State

White Base B1

| Starting material | %* |
|---|---|
| FRAGRANCE | 0.50 |
| ETHYLENE/PROPYLENE/STYRENE | 2.18 |
| COPOLYMER | |
| MICA | 1.20 |
| SUCROSE ACETATE | 5.00 |
| ISOBUTYRATE | |
| ACRYLATES COPOLYMER | 14.48 |
| ISODODECANE | q.s. for 100 |
| POLYHYDROXYSTEARIC ACID | 0.20 |
| HYDROGENATED | 5.29 |
| POLYISOBUTENE | |
| C₃₀₋₅₀ ALCOHOLS | 2.00 |
| POLYETHYLENE | 11.00 |
| | 100 |

Colored Base B2

| Starting materials | % |
|---|---|
| FRAGRANCE | 0.50 |
| ETHYLENE/PROPYLENE/STYRENE | 2.18 |
| COPOLYMER | |
| MICA | 1.20 |
| SUCROSE ACETATE | 5.00 |
| ISOBUTYRATE | |
| IRON OXIDES CI 77499 | 0.17 |
| ALUMINA | 1.39 |
| TITANIUM DIOXIDE CI 77891 | 3.57 |
| YELLOW 6 LAKE CI 15985 | 0.91 |
| ACRYLATES COPOLYMER | 14.48 |
| ISODODECANE | q.s. for 100 |
| RED 7 CI 15850 | 0.77 |
| POLYHYDROXYSTEARIC ACID | 0.20 |
| BLUE 1 LAKE CI 42090 | 0.02 |
| HYDROGENATED | 5.29 |
| POLYISOBUTENE | |
| C₃₀₋₅₀ ALCOHOLS | 2.00 |
| POLYETHYLENE | 11.00 |
| | 100 |

### Example 3

### Matt Formulation in the Liquid State

White Base C1

| Starting material | % |
|---|---|
| OCTYLDODECYL | 0.50 |
| NEOPENTANOATE | |
| BHT | 0.02 |
| TALC | 5.00 |
| ISOSTEARYL ALCOHOL | 0.02 |
| OCTYLDODECANOL | 2.27 |
| ALUMINUM BENZOATE | 0.20 |
| POLYHYDROXYSTEARIC | 0.50 |
| ACID | |
| MALIC ACID | 0.01 |
| DIMETHICONE | 17.35 |
| ARACHIDYL | 5.00 |
| PROPIONATE | |
| DIISOSTEARYL MALATE | 2.24 |
| HYDROGENATED | q.s. for 100 |
| POLYISOBUTENE | |
| PPG-5 LANOLIN WAX | 2.27 |
| WATER | 1.12 |
| POLYETHYLENE | 14.40 |
| POLYBUTENE | 4.55 |
| ALUMINUM STARCH | 6.88 |
| OCTENYLSUCCINATE | |
| C₁₈₋₃₆ ACID | 0.80 |
| TRIGLYCERIDE | |
| DISTEARDIMONIUM | 0.80 |
| HECTORITE | |
| | 100 |

Colored Base C2

| Starting material | % |
|---|---|
| OCTYLDODECYL | 0.50 |
| NEOPENTANOATE | |
| BHT | 0.02 |
| IRON OXIDES | 0.80 |
| KAOLIN | 3.00 |
| ALUMINA | 2.69 |
| TALC | 5.00 |
| YELLOW 6 LAKE | 1.01 |
| RED 28 LAKE | 0.50 |
| ISOSTEARYL ALCOHOL | 0.02 |
| RED 7 | 0.64 |
| OCTYLDODECANOL | 2.27 |
| ALUMINUM BENZOATE | 0.20 |
| POLYHYDROXYSTEARIC | 0.50 |
| ACID | |
| MALIC ACID | 0.01 |
| DIMETHICONE | 17.35 |
| ARACHIDYL | 5.00 |
| PROPIONATE | |
| DIISOSTEARYL MALATE | 2.24 |
| HYDROGENATED | q.s. for 100 |
| POLYISOBUTENE | |
| PPG-5 LANOLIN WAX | 2.27 |
| WATER | 1.12 |
| POLYETHYLENE | 14.40 |
| POLYBUTENE | 4.55 |
| ALUMINUM STARCH | 6.88 |
| OCTENYLSUCCINATE | |
| C₁₈₋₃₆ ACID | 0.80 |
| TRIGLYCERIDE | |
| DISTEARDIMONIUM | 0.80 |
| HECTORITE | |
| | 100 |

### Example 4

### Transfer-Free Formulation in the Liquid State

White Base D1

| Starting material | % |
|---|---|
| FRAGRANCE | 0.3 |
| SQUALANE | 10.86 |
| PHENYL TRIMETHICONE | 28.30 |
| OCTYLDODECANOL | 15.39 |
| SILICA DIMETHYL SILYLATE | 4.5 |
| ISODODECANE | 15 |
| POLYBUTENE | 10.65 |
| | 85 ? |

Colored Base D2

| Starting material | % |
|---|---|
| FRAGRANCE | 0.30 |
| SQUALANE | 10.86 |
| MICA | 1.50 |
| IRON OXIDES | 0.08 |
| IRON OXIDES | 0.25 |
| ALUMINA | 1.64 |
| TITANIUM DIOXIDE | 3.16 |
| YELLOW 6 LAKE | 1.08 |
| PHENYL TRIMETHICONE | 20.03 |
| RED 7 | 0.54 |
| OCTYLDODECANOL | 15.39 |
| SILICA DIMETHYL SILYLATE | 4.50 |
| BLUE 1 LAKE | 0.02 |
| POLYBUTENE | 10.65 |
| ISODODECANE | 15.00 |
| | 100 |

### Example 7

Three ranges of compositions in accordance with the invention, each devoted to one makeup performance, mainly wearing, comfort and gloss, are prepared as indicated above.

The user can, through the use of a method of the invention, select and mix a first composition and a second composition which result from the ranges of compositions provided and can thus obtain a specific composition having features of wearing, of comfort and/or of gloss more particularly adjusted to the expected degree or to the expected intensity.

Formulations

1. Wearing formulation

| | | % |
|---|---|---|
| A | ISODODECANE | 70.00 |
| | HYDROGENATED | 16.73 |
| | POLYISOBUTENE | |
| B | TITANIUM DIOXIDE | 2.74 |
| | RED 7 | 0.54 |
| | BLUE 1 LAKE | 0.16 |
| | YELLOW 6 LAKE | 2.58 |
| | IRON OXIDE | 0.25 |
| C | SILICA DIMETHYL SILYLATE | 7.00 |
| | | 100.00 |

2. Nonglossy comfort formulation

| | | % |
|---|---|---|
| A | HYDROGENATED | 86.73 |
| | POLYISOBUTENE | |
| B | TITANIUM DIOXIDE | 2.74 |
| | RED 7 | 0.54 |
| | BLUE 1 LAKE | 0.16 |
| | YELLOW 6 LAKE | 2.58 |
| | IRON OXIDE | 0.25 |
| C | SILICA DIMETHYL | 7.00 |
| | SILYLATE | |
| | | 100.00 |

3. Gloss formulation

| | | % |
|---|---|---|
| A | POLYBUTENE | 42.00 |
| | HYDROGENATED | 16.73 |
| | POLYISOBUTENE | |
| B | C₁₈₋₃₆ ACID TRIGLYCERIDE | 30.00 |
| | TITANIUM DIOXIDE | 2.74 |
| | RED 7 | 0.54 |
| | BLUE 1 LAKE | 0.16 |
| | YELLOW 6 LAKE | 2.58 |
| | IRON OXIDE | 0.25 |
| C | SILICA DIMETHYL | 5.00 |
| | SILYLATE | |
| | | 100.00 |

1. Adjustment of the Gloss

As indicated in the table below, the user, by selecting and by mixing, as first and second compositions, compositions resulting from the “comfort” and “gloss” ranges of compositions and by adjusting their respective proportions, can thus adjust the gloss of her specific composition exactly to the expected intensity.

The measurements of gloss were carried out with a glossmeter after spreading the composition over a contrast chart, with a film thickness of 150 μm and a period of rest of 10 min at 35° C.

These measurements made it possible to demonstrate the improvement in gloss of a conventional formulation when the latter is mixed in different proportions with a gloss formulation.

The range of gloss of this same product extends from 40.88 to 72.12 at the angle of 20°.

| | Formulation | |
|---|---|---|
| Formulation | proportion (%) | Gloss |
| Nonglossy comfort formulation | 100 | 40.88 |
| (2) | | |
| Nonglossy formulation | 75:25 | 47.58 |
| (2)/glossy formulation (3) | | |
| Nonglossy formulation | 50:50 | 63.50 |
| (2)/glossy formulation (3) | | |
| Nonglossy formulation | 25:75 | 70.26 |
| (2)/glossy formulation (3) | | |
| Glossy formulation (3) | 100 | 72.12 |

2. Adjustment of the Wearing

As indicated in the table below, the user, by selecting and by mixing, as first and second compositions, compositions resulting from “wearing” and “comfort” ranges of compositions and by adjusting the respective proportions, can thus adjust the wearing of her specific composition exactly to the expected intensity.

The wearing is Evaluated as Indicated Above:

These measurements have made it possible to demonstrate the improvement in the wearing of a conventional formulation when the latter is mixed in different proportions with a wearing formulation. The range of wearing of this same product extends from 33 to 78% of wearing after testing.

| | Formulation | |
|---|---|---|
| Formulation | proportion (%) | Wearing |
| Wearing formulation (1) | 100 | 33.43 |
| Wearing formulation | 75:25 | 49.98 |
| (1)/nonglossy comfort | | |
| formulation (2) | | |
| Wearing formulation | 50:50 | 52.57 |
| (1)/nonglossy comfort | | |
| formulation (2) | | |
| Wearing formulation | 25:75 | 53.96 |
| (1)/nonglossy comfort | | |
| formulation (2) | | |
| Nonglossy comfort | 100 | 78.09 |
| formulation (2) | | |

The measurements of wearing carried out for the mixtures (75:25, 50:50 and 25:75) are very similar but make it possible to establish a trend of an improvement in the wearing or less by mixing 50% of each type of formulation.

3. Adjustment of the Color

Gloss Formulation which is a Mixture of Two Hues in Order to Compose a Gammut of Colors from Pink to Red:

The formulations with the compositions indicated below can allow the user, by mixing in specific proportions, to obtain a palette of hues from pink to red in color, making it possible to obtain the desired color intensity.

These compositions, which may or may not be combined, can be used as a mixture with the wearing, comfort and gloss compositions indicated above.

a - Red hue

| Starting material | % |
|---|---|
| SQUALANE | 3.86 |
| BHT | 0.06 |
| DC RED 7 | 1.89 |
| ISOSTEARYL ALCOHOL | 0.32 |
| TRIOLEYL PHOSPHATE | 0.83 |
| ISOPROPYLPARABEN | 0.13 |
| ISOBUTYLPARABEN | 0.10 |
| OCTYLDODECANOL | 8.28 |
| ETHYLHEXYL METHOXYCINNAMATE | 6.99 |
| GLYCERIN | 6.21 |
| MALIC ACID | 0.10 |
| TRIHEPTANOIN | 3.16 |
| PENTAERYTHRITYL TETRAISOSTEARATE | 6.10 |
| DIISOSTEARYL MALATE | q.s. for 100 |
| SILICA DIMETHYL SILYLATE | 6.21 |
| POLYGLYCERYL-2 TRIISOSTEARATE | 8.86 |
| SILICA | 6.18 |
| BEESWAX | 5.66 |
| BUTYLPARABEN | 0.10 |
| C₂₃₋₄₃ ACID PENTAERYTHRITOL TETRAESTER | 0.79 |
| TOTAL | 100 |

b - Pink hue

| Starting material | % |
|---|---|
| SQUALANE | 3.86 |
| MICA | 0.63 |
| BHT | 0.06 |
| IRON OXIDES | 4.00 |
| TITANIUM DIOXIDE | 0.20 |
| TIN OXIDE | 0.01 |
| ISOSTEARYL ALCOHOL | 0.32 |
| TRIOLEYL PHOSPHATE | 0.83 |
| ISOPROPYLPARABEN | 0.13 |
| ISOBUTYLPARABEN | 0.10 |
| OCTYLDODECANOL | 8.28 |
| ETHYLHEXYL METHOXYCINNAMATE | 6.99 |
| GLYCERIN | 6.21 |
| MALIC ACID | 0.10 |
| TRIHEPTANOIN | 3.16 |
| PENTAERYTHRITYL TETRAISOSTEARATE | 6.10 |
| DIISOSTEARYL MALATE | q.s. for 100 |
| SILICA DIMETHYL SILYLATE | 6.21 |
| POLYGLYCERYL-2 TRIISOSTEARATE | 8.86 |
| SILICA | 6.18 |
| BEESWAX | 5.66 |
| BUTYLPARABEN | 0.10 |
| C₂₃₋₄₃ ACID PENTAERYTHRITOL TETRAESTER | 0.79 |
| TOTAL | 100 |

1. A method for making up the lips comprising the stages consisting in:
selecting at least one composition, referred to as “first composition”, from a range of first compositions, said first compositions being formulated in the liquid state, and differing one from each other by a makeup performance other than a color and/or optical effect,
selecting at least one composition, referred to as “second composition”, from a range of second compositions, said range of second compositions being distinct from the range of first compositions,
mixing, at ambient temperature, the first and second compositions selected in the preceding stages, and
applying said mixture to the lips.

2. The method as claimed in claim 1, in which said performance makeup is chosen from a wearing, a non-transfer, a comfort, a glossy, a mattness, a coverage, a moisturizing effect, a desquamating effect, or a cleaning effect type performance.

3. The method as claimed in claim 1, in which the second compositions are formulated in the liquid state.

4. The method as claimed in claim 1, in which the second compositions are formulated in the solid and/or semisolid state.

5. The method as claimed in claim 1, in which a composition referred to as “first composition” has a transfer value of less than or equal to 30 and/or a wearing index of greater than or equal to 60%.

6. The method as claimed in claim 1, in which the mixture applied to the lips has a transfer value of less than or equal to 30 and/or a wearing index of greater than or equal to 60%.

7. The method as claimed in claim 1, in which a composition referred to as “first composition” comprises a volatile compound and/or a film-forming agent in an amount sufficient to provide at least a makeup performance of transfer-free and/or wearing type.

8. The method as claimed in claim 1, in which the volatile compound is a volatile oil chosen from hydrocarbon oils having from 8 to 16 carbon atoms, volatile silicones, volatile fluorinated oils, and their mixtures.

9. The method as claimed in claim 1, in which the composition referred to as “second composition” comprises at least one material capable of providing a color and/or optical effect.

10. The method as claimed in claim 9, in which said material is at least one goniochromatic coloring agent.

11. The method as claimed in claim 9, in which said material is at least one diffractive pigment.

12. The method as claimed in claim 9, in which said material comprises reflective or colored particles visible to the naked eye, in particular glitter or fibers.

13. The method as claimed in claim 1, in which a composition referred to as “second composition” comprises at least one coloring agent chosen from inorganic pigments, organic pigments or lakes, pearlescent pigments, composite pigments, fat-soluble or water-soluble dyes, and their mixtures.

14. The method as claimed in claim 1, in which a first and/or a second composition comprises at least one fatty phase.

15. A range of first and/or second compositions which can be employed in a method as defined in claim 1.
